(19) United States
(12) Patent Application Publication
(10) Pub. No.: US 2025/0349402 A1
(43) Pub. Date: Nov. 13, 2025

(54) METHOD AND APPARATUS FOR MATCHING ANONYMOUS VITALS DATA TO A BLOOD PRODUCT DONOR RECORD

(71) Applicant: Haemonetics Corporation, Boston, MA (US)

(72) Inventors: Melvin Tan, Boston, MA (US); Tracey Loitz, Boston, MA (US)

(21) Appl. No.: 19/202,567

(22) Filed: May 8, 2025

Related U.S. Application Data
(60) Provisional application No. 63/644,291, filed on May 8, 2024.

(51) Int. Cl. G16H 10/60; G06F 21/62; G16H 50/30

(52) U.S. Cl. CPC G16H 10/60; G06F 21/6254; G16H 50/30

(57) ABSTRACT

A blood product donation management method includes determining that a donor is at a location associated with a given vitals device at a given time. A blood establishment computer system receives a vitals message with vitals information relating to the donor obtained from the given vitals device, the vitals message being deidentified to include deidentified information about the donor, and is configured to manage donor information and donation processes. A donor identity of the donor is fetched from the donor records database using the location associated with the identifier of the given vitals device and the given time as database indices to identify the donor with donor identification information. The BECS uses a decision-making framework to determine whether the donor can donate blood and transmits a message indicating whether the donor can donate blood.

### CROSS REFERENCE TO RELATED APPLICATIONS

This application claims priority from U.S. Provisional Patent Application No. 63/644,291, filed May 8, 2024, the contents of which are incorporated by reference herein in its entirety as if fully set forth.

### FIELD

Illustrative embodiments of generally relate to blood processing and, more particularly, various embodiments relate to managing plasma donations in a plasma center.

### BACKGROUND

Plasma donation relies on human donors from whom whole blood is extracted and then separated into individual components, such as plasma. Before a donor can proceed with the plasma donation, they must undergo a screening process at the plasma donation center. This screening is based on historical and health information provided by the donor. Typically, this data is analyzed by donor management software and/or evaluated by staff to determine the donor's eligibility for plasma donation.

Donor information required to assess a donor's eligibility to donate plasma is collected from the donor during their visit to the plasma center. At least part of the screening process is performed by trained and qualified plasma center screening staff at the screening booth where a donor's vitals are captured by a vitals device and then stored in memory of the donor software (e.g., a blood product donation management system) to assess the donor's eligibility to donate. After the vitals have been assessed by the donor software, the donor will be advanced to the next step of the donation process at the plasma center.

The process of adding the information from the vitals device into the donor software typically is done manually by the screening staff reading the values from the vitals device's display and entering the displayed values into the appropriate fields on the donor software. A mistake when reading the vitals from the vitals device or a mistake when inputting the results into the donor software could result in a miscalculation of the donor's eligibility to donate, extra time spent by screening staff to recapture and input the vitals into the donor Software again, extra time to document the mistake in the donor Software, and/or the donor being rejected from donating altogether. A mistake also may accept a person who otherwise would be rejected.

The process also requires the plasma center staff to ensure that the data from the vitals device is matched with the correct donor record in the donor software. Currently, the donor identification information must be inputted to input the system and/or a donor record search must be performed by the plasma center staff so that the vitals can be matched with the correct donor when the vitals are sent to the donor software. This process is cumbersome and prone to error.

### SUMMARY OF VARIOUS EMBODIMENTS

In accordance with one embodiment, a blood donation system for use with a plurality of vitals devices located at a physical blood processing site, each vitals device having a unique identifier, the plurality of vitals devices each configured to determine a human physiological measurement including one or more of donor blood pressure, pulse, temperature, and weight, the blood donation system includes an association engine configured to determine that a donor is at a location associated with a given vitals device at a given time, the association engine also configured to produce a vitals message with vitals information of the donor obtained from the given vitals device, the vitals message being deidentified to include deidentified information about the donor, the vitals information including at least one of the human physiological measurements; a blood establishment computer system (BECS) database client operatively configured to access a BECS donor records database accessible by a BECS, the donor records database being indexed and configured with the unique identifiers of the plurality of vitals devices, the BECS database client configured to fetch a donor identity of the donor from the donor records database using a location associated with the identifier of the given vitals device and the given time as database indices to identify the donor, the BECS database client producing donor information, the BECS database client also configured to fetch as a function of the donor identification information, from the donor records database, additional donor information related to the donor; a decision-making framework operatively coupled with the BECS database client, the decision-making framework configured to determine whether the donor can donate blood as a function of the received vitals information and additional donor information; and a messaging client operatively coupled with the decision-making framework, the messaging client configured to transmit across a network, a go/no-go message indicating whether the donor can donate blood.

In some embodiments, the memory controller is further configured to store the received vitals of the donor the donor records database.

In some embodiments, the BECS memory controller automatically stores the vitals of the identified donor in the donor records database.

In some embodiments, the vitals message is not encrypted.

In some embodiments, the messaging client is configured to forward to the BECS the identity of the identified donor, the given vitals device, and the given time in an ID message, the ID message being sent separately from the vitals message.

In some embodiments, the additional information includes one or more of demographic and health history of the donor.

In some embodiments, the BECS is operatively coupled with the BECS database client.

In some embodiments, the system further includes an apheresis device and a vitals device.

In accordance with one embodiment, a blood product donation management method includes determining that a donor is at a location associated with a given vitals device at a given time, the given vitals device being one of a plurality of vitals devices positioned at a physical blood processing site, each vitals device having a unique identifier, the plurality of vitals devices each configured to determine a human physiological measurement including one or more of donor blood pressure, pulse, temperature, and weight; receiving at a BECS, via a network interface across a network, a vitals message with vitals information relating to the donor obtained from the given vitals device, the vitals message being deidentified to include deidentified information about the donor, the vitals information including at least one of the human physiological measurements, the BECS configured to manage donor information and donation processes; accessing a donor records database using the location associated with the given vitals device at the given time, the donor records database being indexed and configured with the unique identifiers of the plurality of vitals devices; fetching a donor identity of the donor from the donor records database using the location associated with the identifier of the given vitals device and the given time as database indices to identify the donor with donor identification information; fetching, from a donor record accessible by the BECS, additional donor information related to the donor as a function of the donor identification information; using a decision-making framework to determine whether the donor can donate blood as a function of the received vitals information and additional donor information; and transmitting across the network, after using the decision-making framework, a go/no-go message indicating whether the donor can donate blood.

In some embodiments, the method further includes storing in the donor records database received vitals of the identified donor.

In some embodiments, the storing comprises automatically storing the vitals of the identified donor in the donor records database.

In some embodiments, the determining includes determining that a monitor associated with the given vitals device is displaying or has displayed a user interface used by the given donor.

In some embodiments, the vitals message is not encrypted.

In some embodiments, the method further includes transmitting to the BECS the identity of the identified donor, the given vitals device, and the given time in an ID message, the ID message being sent separately from the vitals message.

In some embodiments, the method further includes assigning the identified donor at the physical blood processing site to the given vitals device.

In some embodiments, the additional information includes one or more of demographic and health history of the donor.

In some embodiments, the records database includes non-indexed information.

In accordance with one embodiment, a computer program product for use on a computer system for managing blood donation, the computer program product comprising a tangible, non-transient computer usable medium having computer readable program code thereon. The computer readable program code includes program code for determining that a donor is at a location associated with a given vitals device at a given time, the given vitals device being one of a plurality of vitals devices positioned at a physical blood processing site, each vitals device having a unique identifier, the plurality of vitals devices each configured to determine a human physiological measurement including one or more of donor blood pressure, pulse, temperature, and weight; program code for receiving at a BECS, via a network interface across a network, a vitals message with vitals information relating to the donor obtained from the given vitals device, the vitals message being deidentified to include deidentified information about the donor, the vitals information including at least one of the human physiological measurements, the BECS configured to manage donor information and donation processes; program code for accessing a donor records database using the location associated with the given vitals device at the given time, the donor records database being indexed and configured with the unique identifiers of the plurality of vitals devices; program code for fetching a donor identity of the donor from the donor records database using the location associated with the identifier of the given vitals device and the given time as database indices to identify the donor with donor identification information; program code for fetching, from a donor record accessible by the BECS, additional donor information related to the donor as a function of the donor identification information; program code for using a decision-making framework to determine whether the donor can donate blood as a function of the received vitals information and additional donor information; and program code for transmitting across the network, after using the decision-making framework, a go/no-go message indicating whether the donor can donate blood.

In some embodiments, the computer program product further includes program code for storing in the donor records database received vitals of the identified donor.

In some embodiments, the program code for storing comprises program code for automatically storing the vitals of the identified donor in the donor records database.

Illustrative embodiments are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments from the following “Description of Illustrative Embodiments,” discussed with reference to the drawings summarized immediately below.

FIG. 1 schematically shows one implementation of an example plasma donation system as it interacts within a network in accordance with illustrative embodiments.

FIG. 2 schematically shows an example perspective view of a blood/plasma processing system in accordance with illustrative embodiments.

FIG. 3 schematically shows an example plan view of the blood/plasma processing system of FIG. 2.

FIG. 4 schematically shows an example of a disposable set installed within the blood/plasma processing system of FIG. 2 in accordance with illustrative embodiments.

FIG. 5 schematically shows an example donor software system in accordance with illustrative embodiments.

FIG. 6 shows a flow diagram of an example method of receiving and saving donor vitals information from a donation center in accordance with illustrative embodiments.

FIG. 7 is an example block diagram of a device in accordance with illustrative embodiments.

### DESCRIPTION OF ILLUSTRATIVE EMBODIMENTS

In illustrative embodiments, a plasma center obtains anonymous vitals information from a donor and, using matching technology, matches vitals information to a specific donor's record as a function of the donor's location/use of a vitals device at a specific time at the plasma center.

To that end, plasma center staff may use a vitals device at a plasma center to capture vitals from a donor. After acquiring the requisite vitals from the donor, the vitals device will automatically or manually (e.g., via a request message) send the vitals to donor software, across a network (e.g., a local area network or wide area network) without including any donor identifying attributes. Remotely located donor software may receive the vitals at its network interface and extract key data elements. This donor software also may retrieve, from the message, the date and time of use of the specific vitals device used to obtain the vitals information. The donor software then may query a donor record from a donor database, match the vitals data to a specific donor record using information relating to the donor's specific vitals device at the specific time, and store the vitals data in the donor database with the donor record. Accordingly, this technology mitigates the well-known “man in the middle” problem by sending the vitals information without donor identification information.

When the plasma center staff user reviews the donor record from the donor software database, the donor's vitals are automatically attached, or added, to the donor record. The matching procedure therefore has information relating to one or more donors, and the times they were at their specifically notified vitals device at the plasma center. Using the time information and vitals device information, the matching procedure can match donors with their record in the donor database. As such, deidentified vitals data may be transmitted without significantly risking a donor's identity. Details of illustrative embodiments are discussed below.

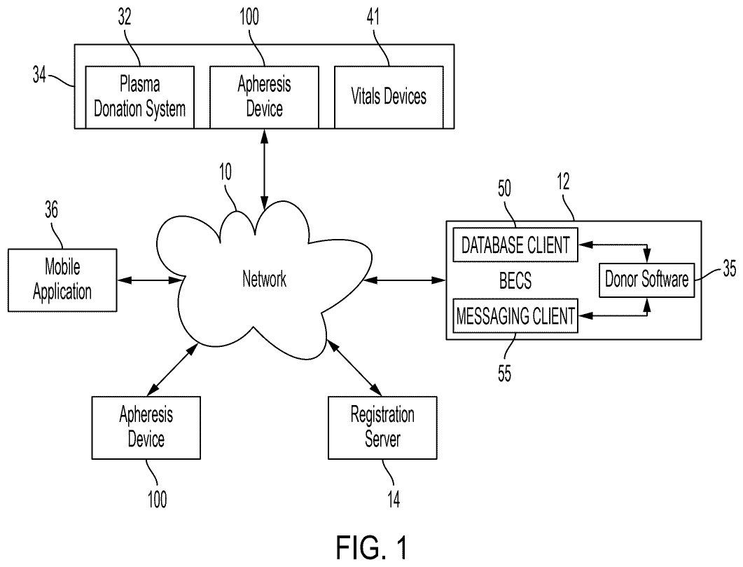

FIG. 1 schematically shows one implementation of a plasma donation system 32 as it interacts with other connected network networked devices in accordance with illustrative embodiments. As shown in this embodiment, the plasma donation system 32 is within a single physical location/site (e.g., a permanent or mobile plasma center 34) with a plurality of on-premises devices. The plasma donation system 32 also may manage and/or collaborate with one or more off-premises devices via a network 10. As shown, the network 10 may include a wide area network (e.g., the Internet), or some other network configuration (e.g., a local area network) communicating a plurality of devices.

A pheresis devices 100 across the network 10 communicate with a registration server 14 in accordance with illustrative embodiments. In this example, two apheresis devices 100 operating within the same functional or business unit are controlled and coordinated by a set of one or more remote device registration servers 14. Among other things, the functional/business entity operating the apheresis devices 100 may be a single plasma center 34. As known by those in the art and discussed in greater detail below, the registration server 14 manages data flow and registration with the apheresis devices 100 and communication with other electronic management systems, such as a remotely located blood establishment computer system (“BECS 12”). In the embodiment shown, the BECS 12 includes donor software 35. For example, the donor software may include features of the Donor360® donor software, distributed by Haemonetics Corporation of Boston, MA. The BECS 12 may be implemented in or across other devices in some embodiments.

In various embodiments, the BECS includes a database client 50 and a messaging client 55. In various embodiments, a database client is a software application to aid interaction with a database server. For example, a database client may execute queries, retrieve data, and manage database objects.

Accordingly, in various embodiments, the database client 50 may access a BECS donor records database accessible by a BECS to fetch a donor identity of a donor from a donor records database using a location associated with an identifier of the given vitals device and the given time as database indices to identify the donor, the BECS database client producing donor information. Additionally, the database client 50 may fetch as a function of donor identification information, from the donor records database, additional donor information related to a donor.

In general, a messaging client is an application that facilitates messages to be transmitted and received over a network. In various embodiments, the messaging client 55 may be utilized to transmit information described in more detail below.

The person who may or will donate plasma (referred to generally as the “donor” or “patient”) may have an associated software application to interact with the plasma donation system 32. For example, the donor may have a mobile telephone or smartphone with a mobile application that interacts with the plasma donation system 32. This mobile application may be used by the donor to augment the donation experience, such as completing required donor questionnaires prior to arrival at the plasma center 34. It also may have geolocation software or other functionality to communicate with the plasma center 34—e.g., a user interface to schedule an appointment.

As noted above, the plasma center 34 also has a plurality of vitals devices 41 to obtain each donor's vitals prior to receiving a donation. Those vitals may include, among other things, blood pressure, pulse, temperature, weight, and protein readings. Based on legal requirements and/or the policy of the donation center, the donor is allowed to donate as a function of those vitals. In illustrative embodiments, the plasma center 34 has a plurality of vitals devices 41 at different locations. In accordance with illustrative embodiments, each vitals device 41 has a prescribed identifier, such as a number, location, and/or name. Accordingly, the plasma center 34 can screen a plurality of different potential donors at the same time.

Indeed, it should be noted that like other figures, FIG. 1 is a simplified figure intended to demonstrate the environment of various embodiments. Those skilled in the art may add further functionality or reduce some functionality shown in that figure. Accordingly, FIG. 1 is illustrative and not intended to limit various embodiments.

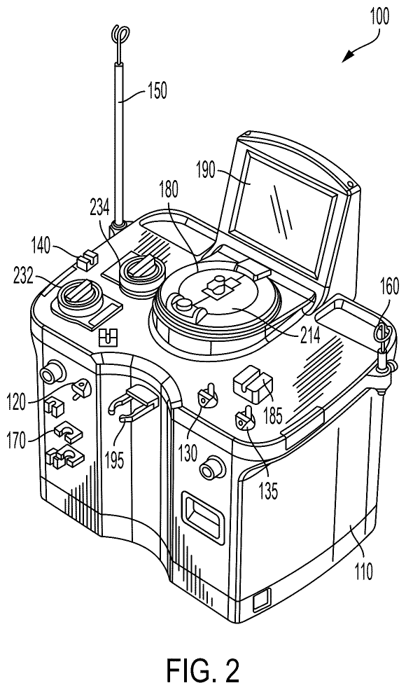

FIGS. 2-4 detail some components making up the network 10 and associated network devices as configured in illustrative embodiments and described above with regard to FIG. 1.

Generally speaking, apheresis devices 100 are medical devices designed to selectively remove specific components from a person's blood while returning the remaining blood components back to the individual. As discussed below in greater detail with regard to FIGS. 2-4, the apheresis device 100 has several primary components. First, it incorporates a system for accessing the individual's blood, often through the insertion of intravenous lines or catheters. The device then uses various methods, such as centrifugation or filtration, to separate the blood components based on their physical or chemical properties. The desired components are collected into specialized containers or bags for further processing or use. Throughout the process, apheresis devices 100 incorporate monitoring systems and control mechanisms to ensure accuracy and safety. These systems may include sensors, pumps, and software interfaces that regulate flow rates, volumes, and other parameters.

Safety features are a crucial aspect of apheresis devices 100. They can include alarms for pressure or flow irregularities, air detection systems, and safety interlocks to protect the donor, patient, and the operator. A pheresis procedures have a wide range of therapeutic applications, including manufacturing into therapies, collecting blood components for transfusion, removing excess or abnormal substances from the blood, and treating specific medical conditions. Plateletpheresis, plasmapheresis, and leukapheresis are examples of therapeutic apheresis procedures.

In medical settings, such as blood or plasma centers, hospitals, and specialized clinics, as well in mobile settings, apheresis devices 100 are employed by trained professionals to perform these procedures. The devices supply the necessary technology and control to efficiently and safely separate blood components. Their use is critical for addressing various therapeutic needs and ensuring the well-being of patients undergoing apheresis procedures.

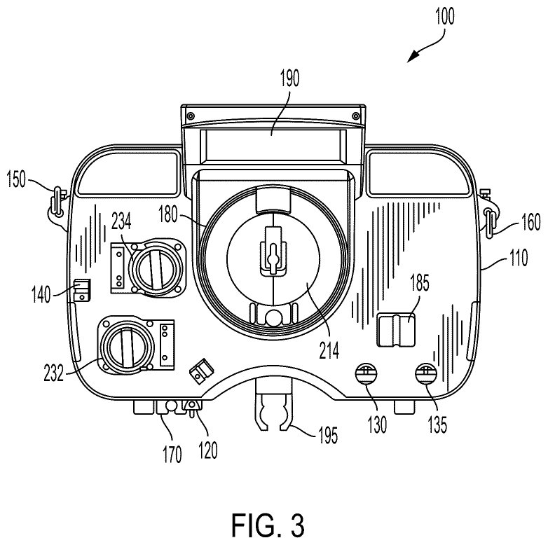

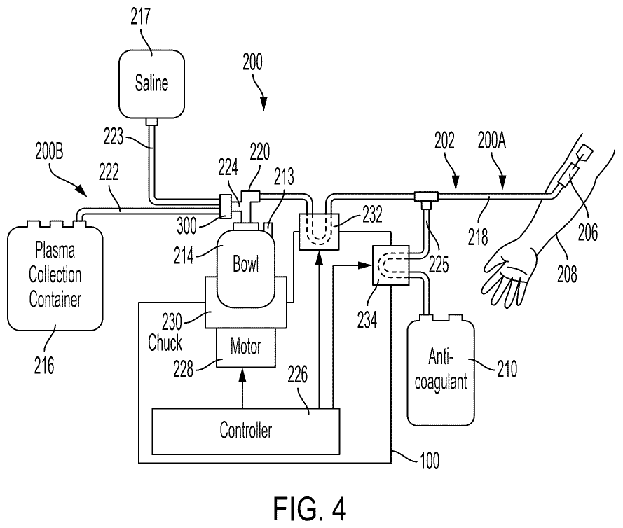

FIG. 2 schematically shows a perspective view of a blood processing system that may be used with illustrative embodiments. FIG. 3 schematically shows a plan view of the blood processing system of FIG. 2. As shown, the blood processing/apheresis device/system 100 includes a cabinet 110 that houses the main components of the system 100 (e.g., the non-disposable components). Within the cabinet 110, the system 100 may include a first/blood pump 232 that draws whole blood from a subject, and a second/anticoagulant pump 234 that pumps anticoagulant through the system 100 and into the drawn whole blood. Additionally, the system 100 may include a number of valves that may be opened and/or closed to control the fluid flow through the system 100. For example, the system 100 may include a donor valve 120 that may open and close to selectively prevent and allow fluid flow through a donor line 218 (e.g., an inlet line shown in FIG. 4), and a plasma valve 130 that selectively prevents and allows fluid flow through an outlet/plasma line 222 (FIG. 4). Some embodiments may also include a saline valve 135 that selectively prevents and allows saline to flow through a saline line 223.

To facilitate the connection and installation of a disposable set and to support the corresponding fluid containers, the system 100 may include an anticoagulant pole 150 on which the anticoagulant solution container 210 (FIG. 4) may be hung, and a saline pole 160 on which a saline solution container 217 (FIG. 4) may be hung (e.g., if the procedure being performed requires the use of saline). Additionally, in some applications, it may be necessary and/or desirable to filter the whole blood drawn from the subject for processing. To that end, the system 100 may include blood filter holder 170 in which the blood filter (located on the disposable set) may be placed.

As discussed in greater detail below, apheresis systems 100 in accordance with illustrative embodiments withdraw whole blood from a subject (aka “donor”) through a venous access device 206 (FIG. 4) using the blood pump 232. As the system 100 withdraws the whole blood from the subject, the whole blood enters a blood component separation device 214, such as a Latham type centrifuge (other type of separation chambers and devices may be used, such as, without limitation, an integral blow-molded centrifuge bowl, as described in U.S. Pat. Nos. 4,983,158 and 4,943,273). The blood component separation device 214 separates the whole blood into its constituent components (e.g., red blood cells, white blood cell, plasma, and platelets). Accordingly, to facilitate operation of the separation device 214, the system 100 may also include a well 180 in which the separation device 214 may be placed and in which the separation device 214 rotates (e.g., to generate the centrifugal forces required to separate the whole blood).

To allow the user/technician to monitor the system operation and control/set the various parameters of the procedure, the system 100 may include a user interface 190 (e.g., a touch screen device) that displays the operation parameters, any alarm messages, and buttons which the user/technician may depress to control the various parameters. Additional components of the blood processing system 100 are discussed in greater detail below (e.g., in relation to the system operation).

FIG. 4 schematically shows, as a block diagram, the blood processing system 100 and a disposable collection set 200 (with an inlet disposable set 200A and an outlet disposable set 200B) that may be loaded onto/into the blood processing system 100, in accordance with the illustrative embodiments. The collection set 200 includes a venous access device 206 (e.g., a phlebotomy needle) for withdrawing blood from a donor's arm 208, a container of anti-coagulant 210, a centrifugation bowl 214 (e.g., a blood component separation device), a saline container 217, and a final plasma collection bag 216. The blood/inlet line 218 couples the venous access device 206 to an inlet port 220 of the bowl 214, the plasma/outlet line 222 couples an outlet port 224 of the bowl 214 to the plasma collection bag 216, and a saline line 223 connects the outlet port 224 of the bowl 214 to the saline container 217. An anticoagulant line 225 connects the anti-coagulant container 210 to the inlet line 218. In addition to the components mentioned above and as shown in FIG. 4, the blood processing system 100 includes a controller 226, a motor 228, and a centrifuge chuck 230. The controller 226 is operably coupled to the two pumps 232 and 234, and to the motor 228, which, in turn, drives the chuck 230. The controller 226 may be operably coupled to and in communication with the user interface 190.

In operation, the disposable collection set 200 (e.g., the inlet disposable set 200A and the outlet disposable set 200B) may be loaded onto/into the blood processing system 100 prior to blood processing. In particular, the blood/inlet line 218 is routed through the blood/first pump 232 and the anticoagulant line 225 from the anti-coagulant container 210 is routed through the anticoagulant/second pump 234. The centrifugation bowl 214 may then be securely loaded into the chuck 230. After the bowl 214 is secured in place, the technician may install the outlet disposable set 200B. For example, the technician may connect a bowl connector 300 to the outlet 224 of the bowl 214, install the plasma container 216 into the weight senor 195, run the saline line 223 through valve 135, and run the plasma/outlet line 222 through valve 130 and the line sensor 185. After the disposable set 200 is installed and the anticoagulant and saline containers 210/217 are connected, the system 100 is ready to begin blood processing.

A pheresis devices 100 are utilized with donors or patients in a controlled and monitored environment, ensuring the safety and well-being of the individual undergoing the procedure. The process typically begins with careful donor or patient preparation and assessment to determine their eligibility and suitability for apheresis.

Before the procedure, the donor's or patient's vital signs, medical history, and relevant laboratory tests are reviewed to ensure that they meet the specific criteria for apheresis. The donor or patient is informed about the procedure, its purpose, and any potential risks or side effects. Informed consent is obtained to ensure that the donor or patient understands the nature of the procedure and provides their agreement to proceed.

After the donor or patient is prepared, an appropriate blood access point is established to facilitate the collection and return of blood. This may involve the insertion of one or more intravenous lines or catheters, depending on the specific requirements of the apheresis procedure. The access points are carefully chosen to minimize discomfort and ensure adequate blood flow during the process.

Next, the apheresis device 100 is set up and configured based on the prescribed parameters for the procedure. This includes programming the device with the desired settings, such as flow rates, separation protocols, and collection volumes, which are tailored to the individual patient's needs.

During the procedure, the apheresis device 100 carefully extracts blood from the donor or patient through the established blood access point. The blood flows through the device, where, as noted with regard to FIGS. 2-4, it undergoes the separation process, either by centrifugation or filtration. The targeted blood component, such as platelets, plasma (in preferred embodiments), or white blood cells, is selectively collected while the remaining blood components are returned to the patient.

Throughout the procedure, the patient's vital signs, including blood pressure, heart rate, and oxygen saturation, are closely monitored to ensure their safety and well-being. The apheresis device's monitoring systems continuously assess critical parameters, such as flow rates, pressures, and component levels, allowing operators to make real-time adjustments if necessary.

Once the desired amount of the targeted component is collected or the prescribed procedure time is reached, the apheresis device 100 completes the process. The donor's or patient's blood access points are carefully removed, and appropriate post-procedure care and monitoring are provided to ensure their comfort and recovery.

The collected blood components often undergo further processing, testing, and preparation as required for their intended therapeutic use. This can involve additional steps such as component labeling, manufacturing, storage, and compatibility testing to ensure their safety and effectiveness when administered to patients.

As noted, the apheresis devices 100 communicate and are controlled in the network 10 via the noted registration server 14. Unlike a typical network server, the registration server 14 has a special role in the apheresis process. Specifically, the registration server 14 in various embodiments acts as a centralized system that facilitates the registration, monitoring, and/or connectivity of apheresis devices 100 from the same locations and/or remote locations. As such, the registration server 14 streamlines the registration and connectivity process for apheresis devices 100 located at various static and/or mobile sites. It further provides a standardized mechanism for registering and identifying individual devices, allowing users (e.g., providers) to have a comprehensive view of the devices within their network 10.

Operation of the apheresis device 100 may be performed in either disconnected mode or connected mode. In the disconnected mode, the apheresis device 100 does not receive or send data to any network enabled computer software system or device. Instead, data collected by the apheresis device 100 remains on the device itself. In connected mode, however, the apheresis device 100 can receive and send data to network enabled computer software systems or devices. The flow of data is bi-directional, meaning it can both send and receive data from external systems.

As noted above, apheresis devices 100 from a single or related unit (e.g., a plasma center 34) can be physically located in different facilities or clinical settings (e.g., see FIG. 1). The registration server 14 therefore acts as a central hub that enables remote management and monitoring of these devices. In addition, the registration server 14 may track assets and manage device activation in case devices 100 are lost or stolen. Registration may involve receiving device-specific information, such as model numbers, activation codes, license information, unique identifiers, and location details. The registration process ensures that each device is properly activated, licensed, authorized, and integrated into the software system, facilitating seamless communication and control. Importantly, the registration server 14 enables remote connectivity and communication with registered apheresis devices 100. It provides a secure and reliable means for remote operators or administrators to access and manage the devices. Through this connectivity, operators can monitor device status, review operational parameters, and perform remote troubleshooting or maintenance tasks as needed. The registration server 14 thus can act as the bridge that facilitates the exchange of data and commands between the remote operators and the apheresis devices 100.

The registration server 14 can be implemented in different ways, depending on the specific setup and requirements of the apheresis devices 100 and the organization. For example, the registration server 14 can be located on the same site as the apheresis devices 100, remotely hosted in an off premises (e.g., in a secure data center), or a combination of both. On-site implementations can facilitate direct and immediate access to these and other devices from the local network 10. Operators or administrators can register and manage the devices within the same physical location, facilitating efficient communication and control. In certain scenarios, a combination of both local and remote configurations may be implemented. This hybrid approach may involve a local registration server 14 at each site where apheresis devices 100 are present, while also connecting to a central remote registration server 14 for higher-level management and coordination. In some embodiments, this setup allows for a balance between local control and centralized oversight. Other embodiments facilitate centralized oversight of the various devices 100 (e.g., a single entity having oversight of many devices 100 across may locations).

The choice of deployment configuration depends on factors, such as the number and distribution of apheresis devices 100, licensing and activation requirements, the organization's IT infrastructure, connectivity requirements, and security considerations. One goal is to ensure seamless connectivity, effective device management, and reliable access to the apheresis devices 100, regardless of whether the registration server 14 is on-site, remote, or a combination of both.

The apheresis device 100 preferably is configured to operate in the connected mode and to automatically connect with the registration server 14. When the apheresis device 100 connects to the Internet (whether through the noted network of FIG. 1 or some other connection), it will automatically register itself with the registration server 14 by sending data about itself, including specific identification data such as serial identifier, location, timestamps, component versions, or any relevant device data such as procedure data, user, or location specific information. After receipt, the registration server 14 preferably processes, stores, and provides a response to the apheresis device 100. This response may include a simple confirmation of device registration, activation codes, licenses, delivery of update files, programming instructions, or new messages received from an external network-enabled system, such as the noted BECS 12 or other networked device. A BECS 12 is shown here as one exemplary implementation.

As known by those in the art, the BECS 12 typically plays a significant role in the optimization of blood banking and plasma collection operations. Designed specifically for blood establishments, these specialized systems offer comprehensive functionalities to manage the collection, testing, processing, storage, and distribution of blood and blood products. When appropriately configured, the BECS 12 can often ensure the safety, traceability, and efficiency of the blood and blood products supply chain.

An important aspect of the BECS 12 in some embodiments involves donor management. This device favorably facilitates the registration and screening of donors, capturing crucial demographic information and medical history. It tracks the lifecycle of a donor's engagement, including donation records, deferrals, and notifications for repeat donations. With robust donor management capabilities, blood establishments can maintain accurate and up-to-date donor data, ensuring the eligibility and safety of the donated blood products.

Another important structure within the BECS 12 involves component management. This functionality allows blood establishments (e.g., a plasma center 34) to track the processing and manufacturing of various blood components, such as red blood cells, platelets, plasma, and cryoprecipitate. By closely monitoring the inventory and location of these components, the BECS 12 enables efficient allocation and usage, minimizing waste and ensuring optimal stock rotation. Additionally, component management ensures proper labeling and tracking, enabling swift retrieval and traceability when needed.

Quality control and testing are integral parts of any plasma center operation, and the BECS 12 streamlines these processes. The system typically integrates laboratory testing results, including compatibility testing, infectious disease screening, and component quality assessment. Test results are securely stored within the BECS 12, facilitating quick access and retrieval when needed. Moreover, the system manages the inventory of test kits and reagents, ensuring their availability and tracking expiration dates. By automating testing and quality control procedures, the BECS 12 enhances accuracy, efficiency, and regulatory compliance.

Reporting and regulatory compliance also form an important aspect of plasma operations. To that end, the BECS 12 generates a wide range of reports, including blood and plasma inventory, donor statistics, adverse events, and performance indicators. These reports not only provide valuable insights for internal management, while also aiding in complying with national and international standards and guidelines. The BECS 12 thus serves as a reliable tool for blood and plasma collection establishments to demonstrate adherence to regulatory requirements and continuously monitor and improve their operations. Some of these functions can cooperate with the apheresis device 100 and registration server 14 to appropriately and effectively complete blood donation and processing.

In the embodiment shown, the registration server 14 acts as a middleware type software element (“middleware”) in the network 10 between the BECS 12 and the apheresis device 100. To that end, the registration server 14 preferably has a middleware engine acting as a bridge, facilitating communication and data exchange between the apheresis devices 100 and the BECS 12. As known by those in the art, middleware engines (also known as “interface engines”) are configured to handle data integration and interoperability between different systems that may use different communication protocols or have incompatible interfaces. They act as a mediator, translating the data formats, protocols, and commands used by the BECS 12 and the apheresis device 100, enabling seamless communication.

Some embodiments may use one or more of the following steps when employing middleware or interface engines:
Integration Configuration: The middleware or interface engine is configured to understand the data formats and communication protocols used by both the BECS 12 and the apheresis device 100. This configuration allows the middleware to act as a translator between the two systems.
Data Translation: The middleware translates the data from the BECS 12 into a format that the apheresis device 100 can understand, and vice versa. This involves mapping the data elements, converting data structures, and ensuring compatibility between the systems.
Protocol Conversion: If the BECS 12 and the apheresis device 100 use different communication protocols, the middleware converts the data transmission to align with the protocols supported by both systems. This ensures that the data can be successfully transmitted and received by each system.
Command Routing: The middleware receives commands from the BECS 12 and routes them to the apheresis device 100. It ensures that the commands are properly formatted and translated to the appropriate commands understood by the apheresis device 100.

Similarly, the middleware receives data and status updates from the apheresis device 100 and forwards them to the BECS 12.

By utilizing a middleware or interface engine, plasma centers 34 can achieve interoperability between their BECS 12 and apheresis devices 100 that may have different communication capabilities or protocols. This approach enables seamless data transfer, command execution, and status monitoring between the two systems.

Middleware or interface engines often adhere to industry standards for healthcare data exchange, such as Health Level Seven (HL7) or Integrating the Healthcare Enterprise (IHE). These standards ensure compatibility and interoperability across different systems within the healthcare domain, facilitating smooth integration and communication between the BECS 12 and the apheresis device 100.

Alternative embodiments may use other approaches for interconnecting the elements of the network 10. For example, in some cases, the BECS 12 may have built-in capabilities to directly integrate with compatible apheresis devices 100. This integration may be achieved through standardized communication protocols or interfaces. The BECS 12 sends commands and receives data directly from the apheresis device 100, allowing for seamless data exchange and control. Other embodiments may have device-specific interfaces. For example, apheresis devices 100 often have their own software interfaces or application programming interfaces (A Pls) that enable communication with external systems like the BECS 12. These interfaces provide a standardized way for the BECS 12 to interact with the apheresis device 100, allowing for data transfer, command execution, and status updates. In either case, some embodiments provide the desired plug-and-play capability for the apheresis devices 100.

Accordingly, discussion of the specific interconnections, as well as some of the devices above (e.g., the BECS 12) are illustrative and not intended to limit various embodiments.

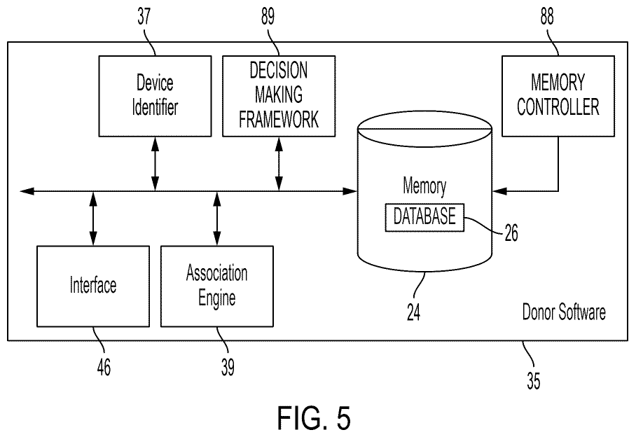

FIG. 5 schematically shows selected components of the donor software 35 of FIG. 1 in accordance with illustrative embodiments. Each of these components is operatively connected by any conventional interconnect mechanism-FIG. 5 simply shows a bus communicating each the components. Those skilled in the art should understand that this generalized representation can be modified to include other conventional direct or indirect connections. Accordingly, discussion of a bus is not intended to limit various embodiments.

Indeed, FIG. 5 only schematically shows selected components/parts of the donor software 35. Those skilled in the art should understand that each of these components can be implemented in a variety of conventional manners, such as by using hardware, software, or a combination of hardware and software, across one or more other functional components. For example, the association engine (to be discussed) may be implemented using a plurality of microprocessors executing firmware. As another example, the association engine may be implemented using one or more application specific integrated circuits (i.e., “ASICs”) and related software, or a combination of ASICs, discrete electronic components (e.g., transistors), and microprocessors. Accordingly, the representation of the association engine and other components in a single box of FIG. 5 is for simplicity purposes only. In fact, in some embodiments, the association engine and/or other parts shown in FIG. 5 can be distributed across a plurality of different machines—not necessarily within the same housing or chassis.

It should be reiterated that the FIG. 5 is a significantly simplified representation of the donor software 35. Those skilled in the art should understand that such a device has many other physical and functional components, such as central processing units, other communication processing modules, and short-term memory. Accordingly, this discussion is not intended to suggest that FIG. 5 represents all of the elements of the donor software 35.

As shown, the donor software 35 has associated memory 24 (e.g., long-term memory, solid state memory) for storing information, such as data from various data generating devices, vitals data/databases with donor records (e.g., from the vitals devices 41), donor information/records, wait times, and a virtual queue of donors. In addition, the plasma donation system 32 has a device identifier 37 configured to detect the identity of any of the vitals devices 41 at the plasma center, an association engine 39 that associates donors with vitals devices 41, and an interface 46 for receiving and forwarding messages (e.g., messages with vitals information) to and from donor software 35.

It should be reiterated that various components of FIG. 5 may be distributed or on other network devices across the network and as such, discussion of the components of FIG. 5 on the donor software 35 is but one of a variety of anticipated embodiments. For example, the device identifier 37 may be located in the plasma donation system 32.

In various embodiments, the memory 24 of the donor software 35 may include a database 26 for storing database information such as patient/donor records. In various embodiments the donor records database may be indexed and configured with unique identifiers of the plurality of vitals devices 41 shown in FIG. 1. Accordingly, the database 26 may be accessible to the database client 50 described above in FIG. 1.

In various embodiments, the donor software includes a memory controller 88. The memory controller 88 computer system that manages the flow of data between a processor or other components and the memory 24. To ensure that data is efficiently read from and written to the memory, coordinating the timing and sequencing of these operations.

For example, the memory controller 88 manages memory access requests from to the memory 24 such as requests for donor information and/or records. Additionally, the memory controller 88 determines the appropriate memory location and handles the transfer of data.

Also, as shown in FIG. 5, is a decision making framework 89. Generally, the decision making framework 89 ensures the safety and efficiency of blood supply management. For example, the decision making framework 89 may identify, assess, and manage risks associated with blood donation, processing, and transfusion. I In the example shown in FIG. 5, the decision making framework 89 may be coupled with the database client 50 described above, to determine whether the donor can donate blood as a function of received vitals information and additional donor information. In various embodiments, the decision making framework 89 may be coupled with the messaging client 55 to indicate to the messaging client 55 to transmit a message across a network (e.g., a go/no-go message indicating whether the donor can donate blood).

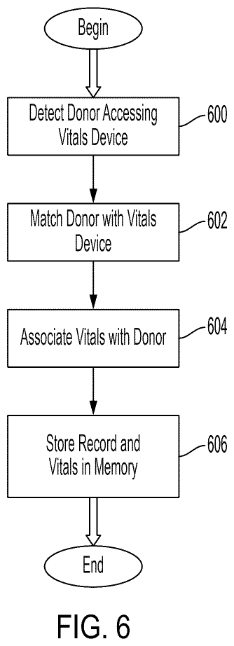

FIG. 6 shows a method of screening plasma donors at a plasma center in accordance with illustrative embodiments. It should be noted that this process is substantially simplified from a longer process that normally would be used. Accordingly, this process may have additional steps that those skilled in the art likely would use. In addition, some of the steps may be performed in a different order than that shown, or at the same time. Those skilled in the art therefore can modify the process as appropriate. Moreover, as noted above and below, the specific processes and structures in each step are exemplary. As such, those skilled in the art can use other processes and/or structures to accomplish the goal of one or more steps, depending upon the application and other constraints. Accordingly, discussion of specific processes and structures is not intended to limit all embodiments.

Before beginning the process, a candidate donor is assigned to have his/her vitals taken at a specific one of the plurality of vitals devices 41 associated with the plasma center. The donor software 35 is aware of this matching—i.e., that this donor candidate is having his/her vitals taken by this specific vitals device 41 at this specific time. This information then may be forwarded to or is otherwise made available to the donor software 35. In some embodiments, geolocation systems can identify the location of the person at a specific vitals device 41. In yet other embodiments, rather than being assigned to a specific vitals device 41, the donor may use any of the vitals devices 41 at the plasma center. The donor may be associated with that vitals machine by any of a number of one or more methods, such as by logging in with their personal information (e.g., via a user interface or scanning a code), accurate location processes via the donor's phone or other device on the donor's person, or other technique. Regardless of the way, the donor and that donor's use information (e.g., times of use) is associated with the specific vitals device 41 he/she is using.

After the vitals device 41 completes its process, that donor's vitals may be displayed on a display device on the vitals device 41 and then forwarded in a message via a non-encrypted channel to the donor software 35 across the network without donor identifying attributes. For example, the message does not include the name, birth date, social security number, or other identification information. Instead, the message may have data or metadata identifying the vitals device 41 used to obtain the vitals information and the time the vitals information was taken for this given donor. Since the information does not include identifying information, the channel may have limited or no encryption, although those in the art and/or regulation may still prefer encryption.

In preferred embodiments, the message with the donor's vitals information is automatically forwarded to the donor software 35 (e.g., across the noted link/channel). In other embodiments, the plasma center staff may initiate a request, from the donor software 35, to send a request to the vitals device 41 to fetch the vitals currently displayed. In another embodiment, the plasma center staff may initiate a request from the vitals device 41 by scanning or inputting donor identifying attributes on the vitals device 41 to automatically initiate a request to send the vitals to the donor software 35 with or without the donor identifying attributes.

At that point, the process of FIG. 6 begins at step 600, in which the device identifier 37 determines that a donor has accessed a given vitals device 41 in the data center at a given time. For example, the device identifier 37, which may be part of or separate from the association engine 39, determines that the noted donor has had her vitals taken at vitals device number 8 between 2:15 μm and 2:30 pm on Jan. 5, 2024. As noted above, this information may have been obtained by a separate interaction, such as by receipt of another message with the donor information and time of the donor use of the vitals device 41.

Armed with this information, the association engine 39 matches the donor vitals with the donor (step 602). To that end, using information in a deidentified message with donor vitals information, the device identifier 37 determines the vitals device 41 and time those vitals were taken from an anonymous donor. Next, using that information, the association engine 39 retrieves use information of the vitals device 41. For example, the use information may include a database of records containing information for each vitals device 41 and a listing and timing of each person/donor using each such vitals device 41. Accordingly, the association engine 39 uses this information to match the anonymous donor with the given vitals device 41 at or after the given time, and then automatically associate one or more received vitals of the donor with the donor record of the matched donor.

Next, the donor software 35 retrieves the donor record of the matched donor and stores or otherwise associates the received vitals with the donor record (step 604). The updated record then is stored in a donor database in memory (step 606).

Plasma center personnel therefore may use this vitals information to determine whether the patient may donate plasma.

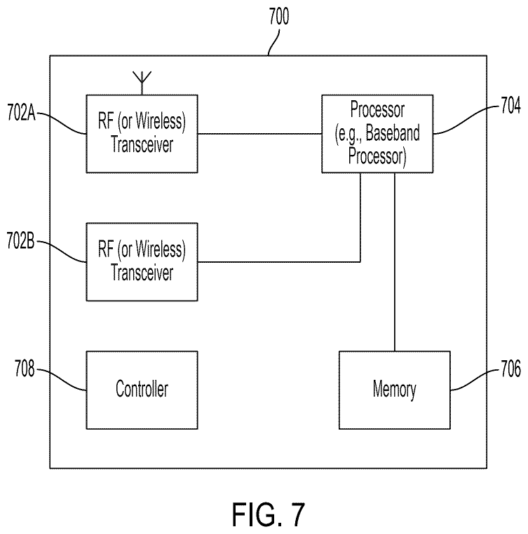

FIG. 7 is an example block diagram of a device 700 in accordance with illustrative embodiments. In various embodiments, the device 700 may be any one of the devices depicted in FIG. 1 and may be a wired or wireless device. For purposes of example, device 700 is depicted as a wireless device.

In various embodiments, the device 700 may include, for example, one or more (e.g., two as shown in FIG. 7) RF (radio frequency) or wireless transceivers 702A, 702B, where each wireless transceiver includes a transmitter to transmit signals and a receiver to receive signals. The device 700 also includes a processor or control unit/entity (controller) 704 to execute instructions or software and control transmission and receptions of signals, and a memory 706 to store data and/or instructions.

Processor 704 may also make decisions or determinations, generate frames, packets or messages for transmission, decode received frames or messages for further processing, and other tasks or functions described herein. Processor 704, which may be a baseband processor, for example, may generate messages, packets, frames or other signals for transmission via wireless transceiver 702 (702A or 702B). Processor 704 may control transmission of signals or messages over a wireless network, and may control the reception of signals or messages, etc., via a wireless network (e.g., after being down-converted by wireless transceiver 702, for example). Processor 704 may be programmable and capable of executing software or other instructions stored in memory or on other computer media to perform the various tasks and functions described above, such as one or more of the tasks or methods described above. Processor 704 may be (or may include), for example, hardware, programmable logic, a programmable processor that executes software or firmware, and/or any combination of these. Using other terminology, processor 704 and transceiver 702 together may be considered as a wireless transmitter/receiver system, for example.

In addition, referring to FIG. 7, a controller (or processor) 708 may execute software and instructions, and may provide overall control for the station 700, and may provide control for other systems not shown in FIG. 7, such as controlling input/output devices (e.g., display, keypad), and/or may execute software for one or more applications that may be provided on wireless device 700, such as, for example, an email program, audio/video applications, a word processor, a Voice over IP application, or other application or software.

In addition, a storage medium may be provided that includes stored instructions, which when executed by a controller or processor may result in the processor 704, or other controller or processor, performing one or more of the functions or tasks described above.

According to another example embodiment, RF or wireless transceiver(s) 702A/702B may receive signals or data and/or transmit or send signals or data. Processor 704 (and possibly transceivers 702A/702B) may control the RF or wireless transceiver 702A or 702B to receive, send, broadcast or transmit signals or data.

Example embodiments are provided or described for each of the example methods, including: An apparatus (e.g., 700, FIG. 7) including means (e.g., processor 704, RF transceivers 702A and/or 702B, and/or memory 706, in FIG. 7) for carrying out any of the methods; a non-transitory computer-readable storage medium (e.g., memory 706, FIG. 7) comprising instructions stored thereon that, when executed by at least one processor (processor 704, FIG. 7), are configured to cause a computing system (e.g., 700, FIG. 7) to perform any of the example methods; and an apparatus (e.g., 700, FIG. 7) including at least one processor (e.g., processor 704, FIG. 7), and at least one memory (e.g., memory 706, FIG. 7) including computer program code, the at least one memory (706) and the computer program code configured to, with the at least one processor (704), cause the apparatus (e.g., 700) at least to perform any of the example methods.

Various embodiments may be implemented at least in part in any conventional computer programming language. For example, some embodiments may be implemented in a procedural programming language (e.g., “C”), or in an object-oriented programming language (e.g., “C++”). Other embodiments may be implemented as a pre-configured, stand-alone hardware element and/or as preprogrammed hardware elements (e.g., application specific integrated circuits, FPGAs, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, solid state drive, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments may be implemented as a combination of both software (e.g., a computer program product) and hardware. Still other embodiments are implemented as entirely hardware, or entirely software.

The embodiments described above are intended to be merely exemplary; numerous variations and modifications will be apparent to those skilled in the art. Such variations and modifications are intended to be within the scope of the present application as defined by any of the appended claims.

What is claimed is:

1. A blood donation system for use with a plurality of vitals devices located at a physical blood processing site, each vitals device having a unique identifier, the plurality of vitals devices each configured to determine a human physiological measurement including one or more of donor blood pressure, pulse, temperature, and weight, the blood donation system comprising:
an association engine configured to determine that a donor is at a location associated with a given vitals device at a given time, the association engine also configured to produce a vitals message with vitals information of the donor obtained from the given vitals device, the vitals message being deidentified to include deidentified information about the donor, the vitals information including at least one of the human physiological measurements;
a BECS database client operatively configured to access a BECS donor records database accessible by a BECS, the donor records database being indexed and configured with the unique identifiers of the plurality of vitals devices, the BECS database client configured to fetch a donor identity of the donor from the donor records database using a location associated with the identifier of the given vitals device and the given time as database indices to identify the donor, the BECS database client producing donor information,
the BECS database client also configured to fetch as a function of the donor identification information, from the donor records database, additional donor information related to the donor;
a decision-making framework operatively coupled with the BECS database client, the decision-making framework configured to determine whether the donor can donate blood as a function of the received vitals information and additional donor information; and
a messaging client operatively coupled with the decision-making framework, the messaging client configured to transmit across a network, a go/no-go message indicating whether the donor can donate blood.

2. The system of claim 1 further comprising BECS memory controller configured to store the received vitals of the donor the donor records database.

3. The system of claim 2 wherein the BECS memory controller automatically stores the vitals of the identified donor in the donor records database.

4. The system of claim 1 wherein the vitals message is not encrypted.

5. The system of claim 1 wherein the messaging client is configured to forward to the BECS the identity of the identified donor, the given vitals device, and the given time in an ID message, the ID message being sent separately from the vitals message.

6. The system of claim 1 wherein the additional information includes one or more of demographic and health history of the donor.

7. The system of claim 1 further comprising a BECS operatively coupled with the BECS database client.

8. The system of claim 1 further comprising an apheresis device and a vitals device.

9. A blood product donation management method comprising:
determining that a donor is at a location associated with a given vitals device at a given time, the given vitals device being one of a plurality of vitals devices positioned at a physical blood processing site, each vitals device having a unique identifier, the plurality of vitals devices each configured to determine a human physiological measurement including one or more of donor blood pressure, pulse, temperature, and weight;
receiving at a BECS, via a network interface across a network, a vitals message with vitals information relating to the donor obtained from the given vitals device, the vitals message being deidentified to include deidentified information about the donor, the vitals information including at least one of the human physiological measurements, the BECS configured to manage donor information and donation processes;
accessing a donor records database using the location associated with the given vitals device at the given time, the donor records database being indexed and configured with the unique identifiers of the plurality of vitals devices;
fetching a donor identity of the donor from the donor records database using the location associated with the identifier of the given vitals device and the given time as database indices to identify the donor with donor identification information;
fetching, from a donor record accessible by the BECS, additional donor information related to the donor as a function of the donor identification information;
using a decision-making framework to determine whether the donor can donate blood as a function of the received vitals information and additional donor information; and
transmitting across the network, after using the decision-making framework, a go/no-go message indicating whether the donor can donate blood.

10. The method of claim 9 further comprising storing in the donor records database received vitals of the identified donor.

11. The method of claim 10 wherein storing comprises automatically storing the vitals of the identified donor in the donor records database.

12. The method of claim 9 wherein determining comprises determining that a monitor associated with the given vitals device is displaying or has displayed a user interface used by the given donor.

13. The method of claim 9 wherein the vitals message is not encrypted.

14. The method of claim 9 further comprising transmitting to the BECS the identity of the identified donor, the given vitals device, and the given time in an ID message, the ID message being sent separately from the vitals message.

15. The method of claim 14 further comprising assigning the identified donor at the physical blood processing site to the given vitals device.

16. The method of claim 9 wherein the additional information includes one or more of demographic and health history of the donor.

17. The method of claim 9 wherein the records database may include non-indexed information.

18. A computer program product for use on a computer system for managing blood donation, the computer program product comprising a tangible, non-transient computer usable medium having computer readable program code thereon, the computer readable program code comprising:
program code for determining that a donor is at a location associated with a given vitals device at a given time, the given vitals device being one of a plurality of vitals devices positioned at a physical blood processing site, each vitals device having a unique identifier, the plurality of vitals devices each configured to determine a human physiological measurement including one or more of donor blood pressure, pulse, temperature, and weight;
program code for receiving at a BECS, via a network interface across a network, a vitals message with vitals information relating to the donor obtained from the given vitals device, the vitals message being deidentified to include deidentified information about the donor, the vitals information including at least one of the human physiological measurements, the BECS configured to manage donor information and donation processes;
program code for accessing a donor records database using the location associated with the given vitals device at the given time, the donor records database being indexed and configured with the unique identifiers of the plurality of vitals devices;
program code for fetching a donor identity of the donor from the donor records database using the location associated with the identifier of the given vitals device and the given time as database indices to identify the donor with donor identification information;
program code for fetching, from a donor record accessible by the BECS, additional donor information related to the donor as a function of the donor identification information;
program code for using a decision-making framework to determine whether the donor can donate blood as a function of the received vitals information and additional donor information; and
program code for transmitting across the network, after using the decision-making framework, a go/no-go message indicating whether the donor can donate blood.

19. The computer program product of claim 18 further comprising program code for storing in the donor records database received vitals of the identified donor.

20. The computer program product of claim 19 wherein the program code for storing comprises program code for automatically storing the vitals of the identified donor in the donor records database.
